(19) United States
(12) Patent Application Publication
(10) Pub. No.: US 2008/0255364 A1
(43) Pub. Date: Oct. 16, 2008

(54) Method For The Production A-Chloroalkylpyridyl Ketones And/Or The Hydrochlorides Thereof

(76) Inventor: Marco Altmayer, Ilvesheim (DE)

Correspondence Address: CONNOLLY BOVE LODGE & HUTZ LLP, 1875 EYE STREET, N.W., SUITE 1100, WASHINGTON, DC, 20036, US

(21) Appl. No.: 11/579,915

(22) PCT Filed: May 17, 2005
(86) PCT No.: PCT/EP2005/005331

§ 371 Date: Nov. 8, 2006

(30) Foreign Application Priority Data
May 18, 2004 (DE) 10 2004 024 565.7

(51) Int. Cl. C07D 213/50

(52) U.S. Cl. 546/315

(57) ABSTRACT

Process for preparing unsubstituted or nuclear-substituted α-chloroalkyl pyridyl ketones and/or their hydrochlorides by reacting the corresponding unsubstituted or nuclear-substituted alkyl pyridyl ketone hydrochlorides with sulfuryl chloride at a reaction temperature of from −25 to 70° C. (248 to 343 K) and a pressure of 0.05 to 0.2 MPa abs, in which the reaction is carried out in the presence of an unbranched or branched C₁ to C₁₀-alkanoic acid which is unsubstituted or monosubstituted to completely substituted by a radical selected from the group of fluorine, chlorine and bromine and whose melting point is below the chosen reaction temperature.

The present invention relates to a process for preparing unsubstituted or nuclear-substituted α-chloroalkyl pyridyl ketones and/or their hydrochlorides by reacting the corresponding unsubstituted or nuclear-substituted alkyl pyridyl ketone hydrochlorides with sulfuryl chloride at a reaction temperature of from −25 to 70° C. (248 to 343 K) and a pressure of from 0.05 to 0.2 MPa abs.

α-Chloroalkyl pyridyl ketones and/or their hydrochlorides are, inter alia, important synthons in the preparation of pharmacologically active ingredients, in particular of β3-adrenoreceptor agonists.

N. J. P. Broom et al., The Journal of Antibiotics, Vol. 48, 1995, No. 11, pages 1336 to 1344 and U.S. Pat. No. 5,561,142 (top of column 17) disclose generally the preparation of α-chloro ketones by reaction of the corresponding carbonyl chlorides with diazo-methane in the presence of hydrogen chloride. The preparation of 3-(2-chloroacetyl)-pyridine hydrochloride and 4-(2-chloroacetyl)pyridine hydrochloride by said synthetic route is described in P. Ribereau et al., Can. J. Chem., Vol. 61, 1983, pages 334 to 342 (see page 339). The disadvantage of this synthetic route is the use of explosive, toxic and carcinogenic diazomethane, which represents a considerable potential hazard and requires elaborate safety measures.

U.S. Pat. No. 5,561,142 discloses generally the preparation of heterocyclic α-chloromethyl ketones by reaction of the corresponding aromatic acetyl compounds with elemental chlorine (see bottom of column 17). The disadvantage of this synthetic route is the use of toxic and corrosive chlorine gas, which requires elaborate safety measures.

U.S. Pat. No. 5,561,142 and U.S. Pat. No. 6,051,586 disclose generally the preparation of heterocyclic α-chloromethyl ketones by reaction of the corresponding aromatic acetyl compounds with N-chlorosuccinimide in the presence of hydrogen chloride and acetic acid (see U.S. Pat. No. 5,561,142, bottom of column 17, and U.S. Pat. No. 6,051,586, bottom of column 11). The synthesis of 3-(2-chloroacetyl)pyridine hydrochloride is described respectively in Examples 14 of U.S. Pat. Nos. 5,561,142 and 5 of U.S. Pat. No. 6,051,586.

J. Duquette et al., Organic Process Research & Development 2003, Vol. 7, No. 3, pages 285 to 288 also discloses the preparation of 3-(2-chloroacetyl)pyridine hydrochloride by said synthetic route. Contrary to the yield of 83% mentioned in the preparation example, the best yield achievable by repetition of a preparation example based on the technical disclosure of the preparation example described in J. Duquette et al. was 20% (see Example 2 in this connection).

The disadvantage of this synthetic route is the low achievable yield, as demonstrated by the abovementioned comparative example. In addition, the use of solid N-chloro-succinimide and its addition to the reaction mixture as solid is disadvantageous. Moreover, N-chlorosuccinimide is a chlorinating agent which is comparatively complicated to prepare and has a correspondingly high price. Furthermore, the synthesis method described in J. Duquette et al. has the disadvantage of slow dropwise addition of liquid 3-acetylpyridine, leading, because of the presence of hydrogen chloride vapors, to the formation of solid 3-acetylpyridine hydrochloride which may block the metering system.

The use in principle of sulfuryl chloride for the α-chlorination of ketones is known per se and described for example in D. P. Wyman et al., J. Org. Chem. Vol. 29, 1964, pages 1956 to 1960.

U.S. Pat. No. 4,310,702 and D. Masilamani et al., J. Org. Chem., Vol. 46, 1981, pages 4486 to 4489, report that the use of sulfuryl chloride for the chlorination of ketones generally leads to a mixture of mono- and polychlorinated ketones and thus to unwanted by-products. The publications disclose the use of alcohols or ethers as moderator to solve the problem.

U.S. Pat. No. 5,710,341, which relates to the preparation of α-chloroalkyl aryl ketones by chlorination of the corresponding ketone with sulfuryl chloride, also discloses the use of aliphatic alcohols for increasing the selectivity for the desired product, i.e. the mono-α-chlorinated ketone.

A disadvantage of the described processes for α-chlorination with sulfuryl chloride is the chlorination of the alcohol employed as side reaction to form alkyl chlorides which may, depending on the molecular weight, be very volatile. Thus, for example, the volatile C₁- to C₃-chloroalkanes are formed in each case from the C₁- to C₃-alkanols mentioned as preferred in U.S. Pat. No. 4,310,702 (bottom of column 1), the methanol and ethanol employed in the examples of U.S. Pat. No. 4,310,702, and the methanol, ethanol and 2-propanol employed in the examples of U.S. Pat. No. 5,710,341. In addition, the solvent methylene chloride used in the examples therein is also very volatile. Since the volatile C₁- to C₃-chloroalkanes and the volatile methylene chloride are harmful to health and the environment, an increased expenditure on off-gas treatment and safety engineering would be necessary on industrial implementation of these processes. The proposed ethers are also generally very volatile compounds, requiring increased expenditure for off-gas treatment and safety engineering.

On the basis of the technical teaching of U.S. Pat. No. 5,710,341, which also specifies, at the top of column 2, that the use of 1-butanol and 2-butanol is particularly preferred, and on the basis of the above-deduced disadvantage of the use of C₁- to C₃-alkanols, the preparation of 3-(2-chloroacetyl)pyridine hydrochloride using 1-butanol, which forms the involatile 1-chlorobutane as byproduct, has been examined experimentally. 3-(2-Chloroacetyl)pyridine hydrochloride was obtained in a yield of only about 51% (see Example 5 concerning this). It emerges from this that although use of the alkanols with a somewhat higher molecular weight, such as, for example, 1-butanol leads to chloroalkanes as byproducts which are easier to handle industrially, the yield of desired product is only very low.

Thus, α-chlorination of alkyl pyridyl ketones with sulfuryl chloride in the presence of an alcohol or ether as moderator in accordance with the teaching described above is disadvantageous because either volatile compounds have to be handled and/or only a low yield of desired product can be obtained.

It is an object of the present invention to find a process for preparing unsubstituted or nuclear-substituted α-chloroalkyl pyridyl ketones and/or their hydrochlorides which does not have the abovementioned disadvantages, avoids the use of explosive or carcinogenic substances, has a high selectivity for monochlorination in the α position and overall makes a high yield of desired product possible.

We have found that this object is achieved by a process for preparing unsubstituted or nuclear-substituted α-chloroalkyl pyridyl ketones and/or their hydrochlorides by reacting the corresponding unsubstituted or nuclear-substituted alkyl pyridyl ketone hydro-chlorides with sulfuryl chloride at a reaction temperature of from −25 to 70° C. (248 to 343 K) and a pressure of from 0.05 to 0.2 MPa abs, wherein the reaction is carried out in the presence of an unbranched or branched C₁- to C₁₀-alkanoic acid which is unsubstituted or monosubstituted to completely substituted by a radical selected from the group of fluorine, chlorine and bromine and whose melting point is below the chosen reaction temperature.

The reaction takes place in the presence of an unbranched or branched C₁- to C₁₀-alkanoic acid which is unsubstituted or monosubstituted to completely substituted by a radical selected from the group of fluorine, chlorine and bromine and whose melting point is below the chosen reaction temperature. The melting point below the chosen reaction temperature ensures that the employed alkanoic acid is also present in liquid form during the reaction.

It is preferred to employ in the process of the invention unbranched C₁- to C₆-alkanoic acids which are unsubstituted or monosubstituted to completely substituted by a radical selected from the group of fluorine and chlorine and whose melting points are below the chosen reaction temperature.

Formic acid, acetic acid, monochloroacetic acid, dichloroacetic acid, trichloroacetic acid, monofluoroacetic acid, difluoroacetic acid, trifluoroacetic acid, propionic acid, butyric acid, pentanoic acid and hexanoic acid are particularly preferably employed, very particularly preferably formic acid, acetic acid, monochloroacetic acid, dichloro-acetic acid, trichloroacetic acid and propionic acid, especially acetic acid.

The alkanoic acid is generally employed in an amount sufficient to make it possible to process and handle the reaction mixture. It is preferred to employ, based on the alkyl pyridyl ketone hydrochloride employed, from 100 to 1000% by weight and particularly preferably from 200 to 400% by weight of alkanoic acid.

In the process of the invention, preferably α-chloroalkyl pyridyl ketones of the general formula (I) and/or their hydrochlorides

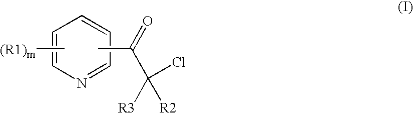

in which
m is 0, 1, 2, 3 or 4;
R¹ is, independently of one another,
   unsubstituted or R⁴-substituted C₁- to C₆-alkyl,
   unsubstituted or R⁴-substituted phenyl,
   unsubstituted or R⁴-substituted C₁- to C₆-alkyloxy,
   unsubstituted or R⁴-substituted phenyloxy,
   unsubstituted or R⁴-substituted C₁- to C₆-acyloxy,
   R⁴, or
   in the case of a position α to the pyridyl nitrogen atom an azide group which is connected to the pyridyl nitrogen atom;
R⁴ is, independently of one another, fluorine, chlorine, bromine, iodine, trifluoromethyl, nitro, cyano, —NR⁵R⁶, —SR⁵, —OR⁵, —SO₂R⁷, —OCOR⁷, —NR⁵COR⁷, —NR⁵SO₂R⁷ or —NR⁵COOR⁶;
R⁵, R⁶, R⁷ is, independently of one another, hydrogen or C₁- to C₆-alky;
R², R³ is, independently of one another, hydrogen or C₁- to C₁₀-alkyl;
are prepared.

The α-chloroalkyl group —CO—CR²R³Cl can be linked in position 2, 3 or 4 on the unsubstituted or nuclear-substituted pyridyl nucleus. It is preferably linked in position 3 on the unsubstituted or nuclear-substituted pyridyl nucleus.

The radicals R² and R³ are preferably independently of one another hydrogen or C₁- to C₆-alkyl, particularly preferably independently of one another hydrogen, methyl, ethyl, 1-propyl, 2-propyl, 1-butyl, 2-butyl, 1-(2-methyl)propyl, 2-(2-methyl)propyl, 1-pentyl or 1-hexyl.

The radicals R⁵, R⁶, R⁷ are preferably independently of one another hydrogen or C₁- to C₄-alkyl, particularly preferably independently of one another hydrogen, methyl, ethyl, 1-propyl, 2-propyl, 1-butyl, 2-butyl, 1-(2-methyl)propyl or 2-(2-methyl)propyl.

If the pyridyl nucleus is unsubstituted, the index m is 0. For substituted pyridyl nuclei, the index m is 1, 2, 3 or 4 depending on whether the pyridyl nucleus is mono-, di-, tri- or tetrasubstituted. The pyridyl nucleus is preferably unsubstituted (m=0), monosubstituted (m=1) or disubstituted (m=2).

It may be emphasized that the radicals R¹ in polysubstituted pyridyl nuclei according to the above definition may be different independently of one another.

The radicals R¹ are preferably independently of one another
unsubstituted or R⁴-substituted C₁- to C₆-alkyl,
unsubstituted or R⁴-substituted phenyl,
unsubstituted or R⁴-substituted C₁- to C₆-alkoxy,
unsubstituted or R⁴-substituted phenyloxy,
unsubstituted or R⁴-substituted C₁- to C₆-acyloxy,
unsubstituted or R⁴-substituted C₁- to C₆-acylamino,
R⁴, or
in the case of a position α to the pyridyl nitrogen atom an azide group which is connected to the pyridyl nitrogen atom;
   where
   R⁴ is, independently of one another, fluorine, chlorine, bromine, iodine, trifluoromethyl, nitro, cyano, —NR⁵R⁶, —OR⁵ or —NR⁵COR⁷;
   R⁵, R⁶, R⁷ is, independently of one another, hydrogen or C₁- to C₆-alkyl.

In the process of the invention, particularly preferably α-chloroalkyl pyridyl ketones of the general formula (II) and/or their hydrochlorides

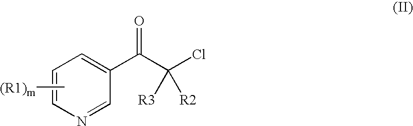

in which
m is 0, 1, 2, 3 or 4;
R¹ is, independently of one another,
unsubstituted or R⁴-substituted C₁- to C₆-alkyl,
unsubstituted or R⁴-substituted phenyl,
unsubstituted or R⁴-substituted C₁- to C₆-alkyloxy,
unsubstituted or R⁴-substituted phenyloxy,
unsubstituted or R⁴-substituted C₁- to C₆-acyloxy,
R⁴, or
in the case of a position α to the pyridyl nitrogen atom an azide group which is connected to the pyridyl nitrogen atom;
   R⁴ is, independently of one another, fluorine, chlorine, bromine, iodine, trifluoromethyl, nitro, cyano, —NR⁵R⁶, —SR⁵, —OR⁵, —SO₂R⁷, —OCOR⁷, —NR⁵COR⁷, —NR⁵SO₂R⁷ or —NR⁵COOR⁶;
   R⁵, R⁶, R⁷ is, independently of one another, hydrogen or C₁- to C₆-alkyl.
   R², R³ is, independently of one another, hydrogen or C₁- to C₁₀-alkyl;
   are prepared.

The radicals R² and R³ are preferably independently of one another hydrogen or C₁- to C₆-alkyl, particularly preferably independently of one another hydrogen, methyl, ethyl, 1-propyl, 2-propyl, 1-butyl, 2-butyl, 1-(2-methyl)propyl, 2-(2-methyl)propyl, 1-pentyl or 1-hexyl.

If the pyridyl nucleus is unsubstituted, the index m is 0. For substituted pyridyl nuclei, the index m is 1, 2, 3 or 4 depending on whether the latter is mono-, di-, tri- or tetra-substituted. The pyridyl nucleus is preferably unsubstituted (m=0), monosubstituted (m=1) or disubstituted (m=2).

It may be emphasized that the radicals R¹ in polysubstituted pyridyl nuclei according to the above definition may be different independently of one another.

The radicals R¹ are preferably independently of one another
unsubstituted or R⁴-substituted C₁- to C₆-alkyl,
unsubstituted or R⁴-substituted phenyl,
unsubstituted or R⁴-substituted C₁- to C₆-alkyloxy,
unsubstituted or R⁴-substituted phenyloxy,
unsubstituted or R⁴-substituted C₁- to C₆-acyloxy,
unsubstituted or R⁴-substituted C₁- to C₆-acylamino,
R⁴, or
in the case of a position α to the pyridyl nitrogen atom an azide group which is connected to the pyridyl nitrogen atom;
   where
   R⁴ is, independently of one another, fluorine, chlorine, bromine, iodine, trifluoromethyl, nitro, cyano, —NR⁵R⁶, —OR⁵ or —NR⁵COR⁷;
   R⁵, R⁶, R⁷ is, independently of one another, hydrogen or C₁- to C₆-alkyl.

Particularly preferred R¹ radicals are methyl, ethyl, 1-propyl, 2-propyl, 1-butyl, 2-butyl, 1-(2-methyl)propyl, 2-(2-methyl)propyl, phenyl, methoxy, ethoxy, 1-propoxy, 2-propoxy, 1-butoxy, 2-butoxy, 1-(2-methyl)propoxy, 2-(2-methyl)propoxy, phenyloxy, formoxy, acetoxy, fluorine, chlorine, acetylamino, propionylamino, butyrylamino, isobutyrylamino, amino, methylamino, ethylamino, 1-propylamino, 2-propylamino, 1-butylamino, 2-butyl-amino, 1-(2-methyl)propylamino, 2-(2-methyl)propylamino and in the case of a position α to the pyridyl nitrogen atom an azide group which is connected to the pyridyl nitrogen atom.

In the process of the invention, very particularly preferably α-chloroalkyl pyridyl ketones of the general formula (II) and/or their hydrochlorides in which

m is 0, 1 or 2;
R¹ is, independently of one another,
fluorine,
chlorine,
—NHCOR⁷ with R⁷ equal to C₁- to C₄-alkyl,
—NR⁵R⁶ with R⁵, R⁶ equal to, independently of one another, hydrogen or C₁- to C₄-alkyl; or
in the case of a position α to the pyridyl nitrogen atom an azide group which is connected to the pyridyl nitrogen atom;
   R⁵, R⁶, R⁷ is, independently of one another, hydrogen or C₁- to C₆-alkyl;
   R², R³ is, independently of one another hydrogen, methyl or ethyl;
   are prepared.

If the pyridyl nucleus is monosubstituted (m=1), α-chloroalkyl pyridyl ketones of the general formula (IIa) and/or their hydrochlorides

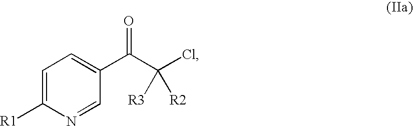

in which R¹, R² and R³ are defined as described above and preferred. If the pyridyl nucleus is disubstituted (m=2), α-chloroalkyl pyridyl ketones of the general formula (IIb) and (IIc) and/or their hydrochlorides

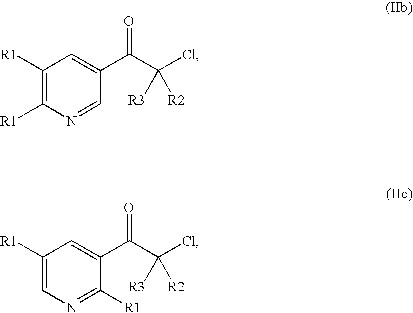

in which R¹, R² and R³ are defined as described above are preferred.

Particularly preferred radicals R¹ are fluorine, chlorine, acetylamino, propionylamino, butyrylamino, isobutyrylamino, amino, methylamino, ethylamino, 1-propylamino, 2-propylamino, 1-butylamino, 2-butylamino, 1-(2-methyl)propylamino, 2-(2-methyl)-propylamino and in the case of a position α to the pyridyl nitrogen atom an azide group which is connected to the pyridyl nitrogen atom.

It is very particularly preferred to prepare in the process of the invention 2-chloro-1-pyridin-3-ylethanone

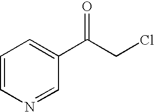

and/or its hydrochloride,
2-chloro-1-(6-chloropyridin-3-yl)ethanone

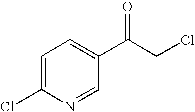

and/or its hydrochloride,
N-[5-(2-chloroacetyl)pyridin-2-yl]isobutyramide

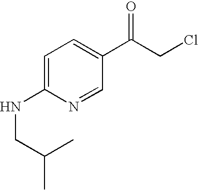

and/or its hydrochloride, or
2-chloro-1-tetrazolo[1,5-a]pyridin-6-ylethanone

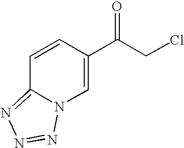

and/or its hydrochloride.

The starting material employed for the reaction in the process of the invention is the appropriate unsubstituted or nuclear-substituted alkyl pyridyl ketone hydrochloride. In this there is a hydrogen atom in place of the α-chloro to be introduced.

The alkyl pyridyl ketone hydrochloride to be employed can for example be added as previously isolated hydrochloride to the reaction mixture or for example be prepared in a preceding reaction by reacting the appropriate alkyl pyridyl ketone with hydrogen chloride, in which case ordinarily no isolation takes place but the reaction mixture is subsequently reacted further according to the invention with sulfuryl chloride in the presence of the defined alkanoic acid. The latter embodiment has the advantage that the generally more easily available alkyl pyridyl ketone can be employed, and no separate isolation and/or purification of the alkyl pyridyl ketone hydrochloride is necessary. It is then more preferred for the employed alkyl pyridyl ketone hydrochloride to be prepared before the addition of sulfuryl chloride by reacting the appropriate alkyl pyridyl ketone with hydrogen chloride.

It is particularly preferred for intermediate preparation of the appropriate alkyl pyridyl ketone by reaction with hydrogen chloride to introduce the alkyl pyridyl ketone into the alkanoic acid and add gaseous hydrogen chloride, particularly preferably by passing into the liquid reaction mixture. The amount of gaseous hydrogen chloride to be added should advantageously correspond at least to the amount required by the stoichiometry. Preferably from 1 to 10 mol, particularly preferably 1 to 5 mol and very particularly preferably 1 to 3 mol of gaseous hydrogen chloride are added per mole of alkyl pyridyl ketone employed.

Irrespective of whether the alkyl pyridyl ketone hydrochloride has been prepared as intermediate as described above or has been added already in the form of the hydrochloride to the reaction mixture, the reaction of the alkyl pyridyl ketone hydrochloride with the sulfuryl chloride in the presence of an alkanoic acid is carried out at a temperature of from −25 to 70° C. (248 to 343 K), preferably 0 to 70° C. (273 to 343 K) and particularly preferably 0 to 50° C. (273 to 323 K). The reaction is carried out under a pressure of from 0.05 to 0.2 MPa abs, preferably 0.09 to 0.2 MPa abs, particularly preferably 0.1 to 0.15 MPa abs and especially under atmospheric pressure.

The sulfuryl chloride is preferably added in liquid and undiluted form while mixing the reaction mixture. The mixing of the reaction mixture takes place for example by stirring. The sulfuryl chloride is generally added according to the progress of the reaction over a period which makes it possible to maintain the desired temperature or the desired temperature range. Since the reaction is exothermic, the reaction vessel is preferably cooled. Depending on the size of the reaction batch, the addition of the sulfuryl chloride lasts minutes or hours. Continuous addition of the sulfuryl chloride is preferred, although a periodic administration is also possible.

The amount of sulfuryl chloride employed is generally from 0.9 to 2 mol, preferably 0.9 to 1.5 mol and particularly preferably 1 to 1.2 mol per mole of alkyl pyridyl ketone hydrochloride employed.

Reaction of the unsubstituted or nuclear-substituted alkyl pyridyl ketone hydrochloride with sulfuryl chloride is also possible in principle in the presence of water because the water which is present initially reacts with the sulfuryl chloride to form sulfuric acid/sulfur trioxide and hydrogen chloride. However, since this involves loss of sulfuryl chloride, it is advantageous to keep the water content of the reaction mixture low. This content is preferably ≦10 mol %, particularly preferably ≦5 mol %, very particularly preferably ≦2 mol % and especially ≦1 mol %, based on the alkyl pyridyl ketone hydrochloride employed.

Because of the desired low water content, preferably low-moisture or anhydrous (concentrated) alkanoic acids are employed. The use of glacial acetic acid is therefore very particularly preferred.

Reaction of the alkyl pyridyl ketone hydrochloride with the sulfuryl chloride in the presence of an alkanoic acid preferably takes place without the addition of further solvents. It is nevertheless possible, however, if appropriate also to employ further solvents such as, for example, chlorinated hydrocarbons, for example dichloromethane, trichloromethane, tetrachloromethane or chlorobenzene. The sulfuryl chloride to be added may also if appropriate be diluted with a solvent and/or the alkanoic acid.

The resulting reaction mixture is, after addition of the sulfuryl chloride is complete, usually mixed further over a period of from several minutes to hours. In order to promote precipitation of the α-chloroalkyl pyridyl ketone hydrochloride formed, it is advantageous if appropriate to cool the mixture. The precipitated α-chloroalkyl pyridyl ketone hydrochloride can be removed from the reaction mixture. This advantageously takes place by filtration, centrifugation or decantation, preferably by filtration or centrifugation. The removed solid is preferably washed with a suitable solvent, for example with an organic ester. The solid can be further purified for example by recrystallization in a suitable solvent, advantageously in an alkanoic acid, then isolated and dried.

If it is desired to prepare the free α-chloroalkyl pyridyl ketone, the latter can be liberated from the resulting α-chloroalkyl pyridyl ketone hydrochloride by reaction with a base. For this purpose, for example the α-chloroalkyl pyridyl ketone hydrochloride is put into a two-phase system comprising water, the base and an organic solvent such as, for example, dichloromethane, methyl tert-butyl ether, toluene or methyltetrahydro-furan. The preferred bases are the readily water-soluble bases such as, for example, sodium hydroxide solution, potassium hydroxide solution, sodium carbonate or potassium carbonate. Generally the pH is adjusted to about 7 to 8 with approximately one equivalent of base per mole of α-chloroalkyl pyridyl ketone hydrochloride. The liberated α-chloroalkyl pyridyl ketone dissolves in the organic phase and can be separated from the aqueous phase by phase separation. The α-chloroalkyl pyridyl ketone can then be isolated from the organic phase, for example by distilling off the solvent.

In a general embodiment, the alkyl pyridyl ketone is introduced into the alkanoic acid, preferably glacial acetic acid, with stirring. Then, to form the alkyl pyridyl ketone hydrochloride, hydrogen chloride is passed into the solution at the desired temperature, if appropriate with cooling. Then, after addition of the hydrogen chloride is complete, the liquid sulfuryl chloride is added, while continuing to stir, the rate of addition having been chosen primarily so that the desired reaction temperature can be maintained and the gas evolution remains controllable. Since the reaction is exothermic, the reaction mixture is generally cooled. After the addition of sulfuryl chloride is complete, the reaction mixture is further stirred over a period of from several minutes to hours. The reaction mixture is preferably cooled during this to a temperature in the range from −25 to 25° C. (248 to 298 K) in order to promote precipitate formation. The precipitated α-chloroalkyl pyridyl ketone hydrochloride is then separated by filtration or centrifugation. Depending on the desired purity, the resulting desired product can be further processed directly in the resulting form or be worked up for purification. If the free α-chloroalkyl pyridyl ketone is desired, it is liberated in a two-phase system comprising water, a base and an organic solvent, and is isolated from the organic phase.

The process of the invention makes it possible to prepare unsubstituted or nuclear-substituted α-chloroalkyl pyridyl ketones and/or their hydrochlorides without employing explosive or carcinogenic substances, has a high selectivity for monochlorination in the α position and overall makes a high yield of desired product possible. Depending on the desired final product, the α-chloroalkyl pyridyl ketone hydrochlorides or, after liberation by a base, the free α-chloroalkyl pyridyl ketones can be prepared in high purity. The sulfuryl chloride to be employed as chlorinating agent is readily available at relatively low-cost especially in relation to other chlorinating agents such as, for example, N-chlorosuccinimide. In addition, sulfuryl chloride can, unlike N-chloro-succinimide, be metered as liquid, which is advantageous in industrial operations. Compared with prior art α-chlorination processes using sulfuryl chloride, the present process of the invention avoids the use of alcohols as moderators because of the presence of an alkanoic acid.

### EXAMPLES

### Example 1

### According to the Invention

### Synthesis of 2-chloro-1-pyridin-3-ylethanone hydrochloride

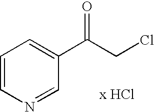

50 g (0.41 mol) of 3-acetylpyridin and 100 g of glacial acetic acid were mixed and cooled to 15° C. 34 g (0.93 mol) of hydrogen chloride gas were passed into the mixture at 15 to 20° C. Then, over the course of 30 minutes at 20 to 25° C., 60.89 g (0.45 mol) of sulfuryl chloride were metered in. This resulted in a white suspension which was stirred for a further 12 hours at room temperature after the sulfuryl chloride metering was complete. Then an additional 50 g of glacial acetic acid was added to the reaction mixture which was then heated to reflux until the suspended solid had completely dissolved. The mixture was subsequently cooled to 15° C. The precipitated required product was filtered off, washed 3 times with 45 g of ethyl acetate each time and finally dried in vacuo at 25° C. 67.8 g (0.353 mol) were obtained, corresponding to a yield of 86.1% of theory.

The following analytical data were obtained:

melting point: 178° C.

chemical purity (after liberation from the hydrochloride): 95 GC area %. For this, the resulting 2-chloro-1-pyridin-3-ylethanone hydrochloride was put into a water/methylene chloride mixture and adjusted to neutral pH with sodium hydroxide solution. The liberated 2-chloro-1-pyridin-3-ylethanone accumulated in the methylene chloride phase, which was analyzed by gas chromatography.

¹³C-NMR (125 MHz, d6-DMSO): 189.4 (C═O); 147.5 (t); 144.0 (t); 142.0 (t); 131.9 (q); 126.5 (t); 47.9 (s).

¹H-NMR (500 MHz, d6-DMSO): 9.4 (1H); 9.1 (1H); 8.9 (1H); 8.1 (1H); 5.5 (2H).

### Example 2

### Comparative Example

This comparative example was based on the technical teaching of the preparation example described in J. Duquette et al., Organic Process Research & Development 2003, Vol. 7, No. 3, pages 285 to 288.

238 ml of glacial acetic acid were introduced into the reaction flask and, over the course of 30 minutes at 15 to 20° C. with ice-bath cooling, 20 g (0.548 mol) of hydrogen chloride gas were passed into the reaction flask above the surface of the liquid. The temperature was then raised to 20° C. and, at this temperature, 25 g (0.207 mol) of 3-acetylpyridine were added dropwise over the course of 30 minutes. Then 29.64 g (0.222 mol) of N-chlorosuccinimide were put into the pale yellow solution, and the resulting yellow solution was stirred at 20 to 25° C. In contrast to the example described in J. Duquette et al., in the present case no precipitate had formed even after 12 hours.

For this reason, the solution was seeded with seed crystals of 2-chloro-1-pyridin-3-yl-ethanone hydrochloride. Even after this, precipitate formed. Thereafter the temperature was lowered to 15 to 20° C. by ice-bath cooling, and 19 g (0.521 mol) of hydrogen chloride gas were passed directly into the solution. Only after a further 30 minutes did a white solid separate out, and was filtered off. The filtered precipitate was washed with ethyl acetate and dried in vacuo at 25° C. The yield of 2-chloro-1-pyridin-3-ylethanone hydrochloride was only 20% of theory.

Example 2 shows that the preparation example described in J. Duquette et al. could not be repeated and led to a quite different result. Even after significant modification in relation to seeding, cooling and renewed passing in of hydrogen chloride gas, only a yield of 20% was achievable.

### Example 3

### Comparative Example

This comparative example was carried out as described in Example 2 with the exception that the hydrogen chloride gas was passed directly into the glacial acetic acid, not above the surface of the liquid. Again, no precipitate had formed after 12 hours even with this experimental procedure. The experiment was terminated at this point.

Even passing hydrogen chloride gas directly into the glacial acetic acid did not lead to a different result from Example 2.

### Example 4

### Comparative Example

12 g (0.099 mol) of 3-acetylpyridine were introduced into 95 ml of glacial acetic acid and, at 20 to 25° C., 16.9 g (0.46 mol) of hydrogen chloride gas were passed in with ice-bath cooling. Subsequently, 12.7 g (0.095 mol) of N-chlorosuccinimide were added in one portion. A white suspension was produced during the subsequent stirring time of 12 hours. The precipitated solid was filtered off, washed with ethyl acetate and dried in a stream of nitrogen. The yield of 2-chloro-1-pyridin-3-ylethanone hydrochloride was 68.42% of theory.

The following analytical data were obtained:

chemical purity (after liberation from the hydrochloride): 89.47 GC area %. Analysis took place as described in Example 1.

Even with a marked modification of the preparation method described in J. Duquette et al., by first adding the 3-acetylpyridine and only then passing in hydrogen chloride gas, it was far from possible to achieve the yield of 83% asserted in J. Duquette et al.

### Example 5

### Comparative Example

This comparative example was based on the technical teaching of the preparation process described in U.S. Pat. No. 5,710,341, employing 3-acetylpyridine hydrochloride in place of the aryl alkyl ketones mentioned.

13.5 g (0.086 mol) of 3-acetylpyridine hydrochloride were introduced into 25.2 g (0.34 mol) of n-butanol and, at 20 to 30° C., 34.8 g (0.258 mol) of sulfuryl chloride were metered in over the course of 15 minutes. After a subsequent stirring time of 2 hours, a sample was taken from the reaction mixture and analyzed by gas chromatography after the sample had been worked up as in Example 1. A conversion (according to GC area %) of 72% and a selectivity of 71.4% for 2-chloro-1-pyridin-3-ylethanone was detected. The yield resulting therefrom is about 51%.

Example 5 shows that the α-chlorination process disclosed in U.S. Pat. No. 5,710,341 leads on use of alkyl pyridyl ketones and/or their hydrochloride to only an inadequately low yield of α-chloroalkyl pyridyl ketones and/or their hydrochlorides.

### Example 6

### According to the Invention

### Synthesis of 2-chloro-1-(6-chloropyridin-3-yl)ethanone hydrochloride

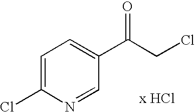

63.0 g (0.405 mol) of 1-(6-chloropyridin-3-yl)ethanone and 142 g of propionic acid were mixed and cooled to 15° C. At 15 to 20° C., 48.0 g (1.32 mol) of hydrogen chloride gas were passed into the mixture. Then, over the course of 30 minutes at 20 to 25° C., 58.05 g (0.43 mol) of sulfuryl chloride were metered in. This resulted initially in a yellow solution. After the sulfuryl chloride metering was complete, the mixture was stirred at room temperature for a further 12 hours, resulting in a beige suspension. The precipitated required product was filtered off and washed with 50 g of propionic acid. For further purification, the moist solid was suspended in 600 g of water and 500 g of methyl tert-butyl ether (MTBE). The mixture was adjusted to pH=6 with about 163 g of 25% strength sodium hydroxide solution. The aqueous phase was separated off and extracted once with 200 g of MTBE. The MTBE phases were combined, washed once with 250 g of water and dried over sodium sulfate. The hydrochloride was precipitated by passing 50 g (1.37 mol) of hydrogen chloride into the dried MTBE phase. The solid which formed was filtered off, washed with 70 g of MTBE and dried in vacuo at 25° C. 56.1 g (0.248 mol) were obtained, corresponding to a yield of 61.2% of theory.

The following analytical data were obtained:

melting point: 106 to 107° C.

chemical purity (after liberation from the hydrochloride): >98 GC area %. Determination as for example 1.

13C-NMR (125 MHz, d6-DMSO): 190.2 (C═O); 154.5 (q); 150.1 (t); 139.2 (t); 129.3 (q); 124.6 (t); 47.6 (s).

1H-NMR (500 MHz, d6-DMSO): 9.0 (1H); 8.4 (1H); 7.8 (1H); 5.3 (2H).

### Example 7

### According to the Invention

### Synthesis of 2-chloro-1-pyridin-3-ylethanone, hydrochloride

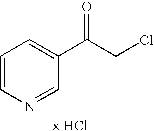

500 g (4.13 mol) of 3-acetylpyridine and 1500 g of propionic acid were mixed and cooled to 15° C. At 15 to 20° C., 340 g (9.32 mol) of hydrogen chloride gas were passed into the mixture. Then, over the course of 160 minutes at 15 to 25° C., 608.9 g (4.51 mol) of sulfuryl chloride were metered in. This resulted in a white suspension which was stirred after the metering of sulfuryl chloride was complete at room temperature for a further 12 hours. The precipitated required product was filtered off, washed 3 times with 500 g of ethyl acetate each time and finally dried in vacuo at 25° C. 773.4 g (4.03 mol) were obtained, corresponding to a yield of 97.5% of theory.

The following analytical data were obtained:

melting point: 178° C.

chemical purity (after liberation from the hydrochloride): >95 GC area %. Determination as in example 1.

melting point: 178° C.

13C-NMR (125 MHz, d6-DMSO): 189.4 (C═O); 147.5 (t); 144.0 (t); 142.0 (t); 131.9 (q); 126.5 (t); 47.9 (s)

1H-NMR (500 MHz, d6-DMSO): 9.4 (1H); 9.1 (1H); 8.9 (1H); 8.1 (1H); 5.5 (2H).

1. A process for preparing unsubstituted or nuclear-substituted α-chloroalkyl pyridyl ketones and/or their hydrochlorides, the process comprising:
reacting the corresponding unsubstituted or nuclear-substituted alkyl pyridyl ketone hydro-chlorides with sulfuryl chloride at a reaction temperature of from −25 to 70° C. (248 to 343 K) and a pressure of from 0.05 to 0.2 MPa abs,
wherein the reaction is carried out in the presence of an unbranched or branched C₁- to C₁₀-alkanoic acid, which is unsubstituted or monosubstituted to completely substituted by a radical selected from the group consisting of fluorine, chlorine and bromine, and whose melting point is below the reaction temperature.

2. The process according to claim 1, wherein the reaction is carried out in the presence of formic acid, acetic acid, monochloroacetic acid, dichloroacetic acid, trichloroacetic acid or propionic acid.

3. The process according to claim 1, wherein the alkanoic acid is employed in an amount of from 100 to 1000% by weight based on the alkyl pyridyl ketone hydrochloride employed.

4. The process according to claim 1, wherein α-chloroalkyl pyridyl ketones of the general formula (I) and/or their hydrochlorides 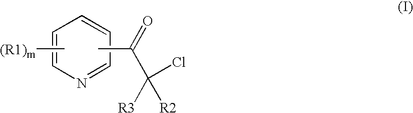
wherein
m is 0, 1, 2, 3 or 4;
R¹ is, independently of one another,
unsubstituted or R⁴-substituted C₁- to C₆-alkyl,
unsubstituted or R⁴-substituted phenyl,
unsubstituted or R⁴-substituted C₁- to C₆-alkyloxy,
unsubstituted or R⁴-substituted phenyloxy,
unsubstituted or R⁴-substituted C₁- to C₆-acyloxy,
R⁴, or
in the case of a position α to the pyridyl nitrogen atom an azide group which is connected to the pyridyl nitrogen atom;
R⁴ is, independently of one another, fluorine, chlorine, bromine, iodine, trifluoromethyl, nitro, cyano, —NR⁵R⁶, —SR⁵, —OR⁵, —SO₂R⁷, —OCOR⁷, —NR⁵COR⁷, —NR⁵SO₂R⁷ or —NR⁵COOR⁶;
R⁵, R⁶, R⁷ is, independently of one another, hydrogen or C₁- to C₆-alkyl; and
R², R¹ is, independently of one another, hydrogen or C₁- to C₆-alkyl;
are prepared.

5. The process according to claim 1, wherein α-chloroalkyl pyridyl ketones of the general formula (II) and/or their hydrochlorides 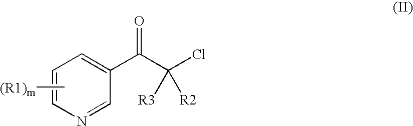
wherein m, R¹, R² and R³ have the same meaning as defined in claim 4, are prepared.

6. The process according to claim 5, wherein α-chloroalkyl pyridyl ketones of the general formula (II) and/or their hydrochlorides, in which wherein
m is 0, 1 or 2;
R¹ is, independently of one another,
fluorine,
chlorine,
—NHCOR⁷ with R⁷ equal to C₁- to C₄-alkyl,
—NR⁵R⁶ with R⁵, R⁶ equal to, independently of one another, hydrogen or C₁- to C₄-alkyl; or
in the case of a position α to the pyridyl nitrogen atom an azide group which is connected to the pyridyl nitrogen atom;
R⁵, R⁶, R⁷ is, independently of one another, hydrogen or C₁- to C₆-alkyl; and
R², R³ is, independently of one another hydrogen, methyl or ethyl;
are prepared.

7. The process according to claim 1, wherein the alkyl pyridyl ketone hydrochloride employed is prepared before the addition of sulfuryl chloride by reacting the alkyl pyridyl ketone with hydrogen chloride.

8. The process according to claim 7, wherein the alkyl pyridyl ketone is reacted with hydrogen chloride by introducing the alkyl pyridyl ketone into the alkanoic acid and adding from 1 to 5 mol of gaseous hydrogen chloride per mole of alkyl pyridyl ketone employed.

9. The process according to claim 1, wherein the sulfuryl chloride is employed in an amount of from 0.9 to 1.5 mol per mole of alkyl pyridyl ketone hydrochloride employed.

10. The process according to claim 1, wherein the α-chloroalkyl pyridyl ketone is liberated by reacting the α-chloroalkyl pyridyl ketone hydrochloride obtained with a base.

11. The process according to claim 1, wherein 2-chloro-1-pyridin-3-ylethanone and/or its hydrochloride, 2-chloro-1-(6-chloropyridin-3-yl)ethanone and/or its hydrochloride, N-[5-(2-chloroacetyl)pyridin-2-yl]isobutyramide and/or its hydrochloride or 2-chloro-1-tetrazolo[1,5-a]pyridin-6-ylethanone and/or its hydrochloride is prepared.

12. The process according to claim 2, wherein the alkanoic acid is employed in an amount of from 100 to 1000% by weight based on the alkyl pyridyl ketone hydrochloride employed.

13. The process according to claim 2, wherein α-chloroalkyl pyridyl ketones of the general formula (I) and/or their hydrochlorides 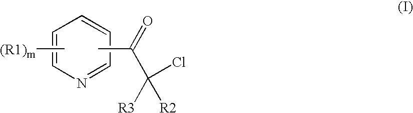
wherein
m is 0, 1, 2, 3 or 4;
R¹ is, independently of one another,
unsubstituted or R⁴-substituted C₁- to C₆-alkyl,
unsubstituted or R⁴-substituted phenyl,
unsubstituted or R⁴-substituted C₁- to C₆-alkyloxy,
unsubstituted or R⁴-substituted phenyloxy,
unsubstituted or R⁴-substituted C₁- to C₆-acyloxy,
R⁴, or
in the case of a position α to the pyridyl nitrogen atom an azide group which is connected to the pyridyl nitrogen atom;
R⁴ is, independently of one another, fluorine, chlorine, bromine, iodine, trifluoromethyl, nitro, cyano, —NR⁵R⁶, —SR⁵, —OR⁵, —SO₂R⁷, —OCOR⁷, —NR⁵COR⁷, —NR⁵SO₂R⁷ or —NR⁵COOR⁶;
R⁵, R⁶, R⁷ is, independently of one another, hydrogen or C₁- to C₆-alkyl; and
R², R³ is, independently of one another, hydrogen or C₁- to C₁₀-alkyl;
are prepared.

14. The process according to claim 3, wherein α-chloroalkyl pyridyl ketones of the general formula (I) and/or their hydrochlorides 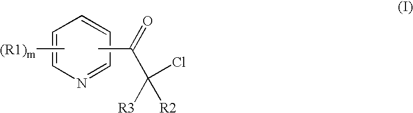
wherein
m is 0, 1, 2, 3 or 4;
R¹ is, independently of one another,
unsubstituted or R⁴-substituted C₁- to C₆-alkyl,
unsubstituted or R⁴-substituted phenyl,
unsubstituted or R⁴-substituted C₁- to C₆-alkyloxy,
unsubstituted or R⁴-substituted phenyloxy,
unsubstituted or R⁴-substituted C₁- to C₆-acyloxy,
R⁴, or
in the case of a position α to the pyridyl nitrogen atom an azide group which is connected to the pyridyl nitrogen atom;
R⁴ is, independently of one another, fluorine, chlorine, bromine, iodine, trifluoromethyl, nitro, cyano, —NR⁵R⁶, —SR⁵, —OR⁵, —SO₂R⁷, —OCOR⁷, —NR⁵COR⁷, —NR⁵SO₂R⁷ or —NR⁵COOR⁶;
R⁵, R⁶, R⁷ is, independently of one another, hydrogen or C₁- to C₆-alkyl; and
R², R³ is, independently of one another, hydrogen or C₁- to C₁₀-alkyl;
are prepared.

15. The process according to claim 2, wherein α-chloroalkyl pyridyl ketones of the general formula (II) and/or their hydrochlorides 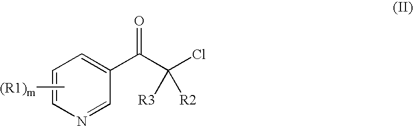
wherein m, R¹, R² and R³ have the same meaning as defined in claim 4, are prepared.

16. The process according to claim 3, wherein α-chloroalkyl pyridyl ketones of the general formula (II) and/or their hydrochlorides 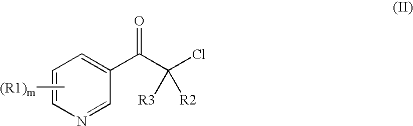
wherein m, R¹, R² and R³ have the same meaning as defined in claim 4, are prepared.

17. The process according to claim 4, wherein α-chloroalkyl pyridyl ketones of the general formula (II) and/or their hydrochlorides 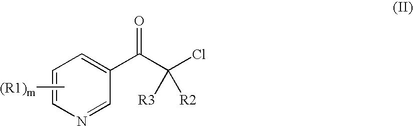
wherein m, R¹, R² and R³ have the same meaning as defined in claim 4, are prepared.

18. The process according to claim 15, wherein α-chloroalkyl pyridyl ketones of the general formula (II) and/or their hydrochlorides, wherein
m is 0, 1 or 2;
R¹ is, independently of one another,
fluorine,
chlorine,
—NHCOR⁷ with R⁷ equal to C₁- to C₄-alkyl,
—NR⁵R⁶ with R⁵, R⁶ equal to, independently of one another, hydrogen or C₁- to C₄-alkyl; or
in the case of a position α to the pyridyl nitrogen atom an azide group which is connected to the pyridyl nitrogen atom;
R⁵, R⁶, R⁷ is, independently of one another, hydrogen or C₁- to C₆-alkyl; and
R², R³ is, independently of one another hydrogen, methyl or ethyl;
are prepared.

19. The process according to claim 16, wherein α-chloroalkyl pyridyl ketones of the general formula (II) and/or their hydrochlorides, wherein
m is 0, 1 or 2;
R¹ is, independently of one another,
fluorine,
chlorine,
—NHCOR⁷ with R⁷ equal to C₁- to C₄-alkyl,
—NR⁵R⁶ with R⁵, R⁶ equal to, independently of one another, hydrogen or C₁- to C₄-alkyl; or
in the case of a position α to the pyridyl nitrogen atom an azide group which is connected to the pyridyl nitrogen atom;
R⁵, R⁶, R⁷ is, independently of one another, hydrogen or C₁- to C₆-alkyl; and
R², R³ is, independently of one another hydrogen, methyl or ethyl;
are prepared.

20. The process according to claim 17, wherein α-chloroalkyl pyridyl ketones of the general formula (II) and/or their hydrochlorides, wherein
m is 0, 1 or 2;
R¹ is, independently of one another,
fluorine,
chlorine,
—NHCOR⁷ with R⁷ equal to C₁- to C₄-alkyl,
—NR⁵R⁶ with R⁵, R⁶ equal to, independently of one another, hydrogen or C₁- to C₄-alkyl; or
in the case of a position α to the pyridyl nitrogen atom an azide group which is connected to the pyridyl nitrogen atom;
R⁵, R⁶, R⁷ is, independently of one another, hydrogen or C₁- to C₆-alkyl; and
R², R³ is, independently of one another hydrogen, methyl or ethyl;
are prepared.
